(19) United States
(12) Patent Application Publication
(10) Pub. No.: US 2024/0269713 A1
(43) Pub. Date: Aug. 15, 2024

(54) ANTI-INFECTION SURGICAL DEVICE AND METHOD FOR BIOFILM AGITATION

(71) Applicants: Timothy McManus, Hanover, NH (US); Ryan Greeley McManus, Hanover, NH (US)

(72) Inventors: Timothy McManus, Hanover, NH (US); Ryan Greeley McManus, Hanover, NH (US)

(21) Appl. No.: 18/438,248

(22) Filed: Feb. 9, 2024

Related U.S. Application Data
(60) Provisional application No. 63/484,115, filed on Feb. 9, 2023.

(51) Int. Cl. B08B 1/30; A46B 9/00; A46B 9/06; A46B 11/06; A46B 13/02; A46B 13/04; A46B 15/00; A46D 1/00; A61B 17/56; A61L 2/04; A61L 2/10; A61L 2/26; B08B 1/12; B08B 1/40; B08B 5/04; B08B 7/00; B08B 7/02

(52) U.S. Cl. CPC B08B 1/30; A46B 9/005; A46B 9/06; A46B 11/063; A46B 13/023; A46B 13/04; A46B 15/003; A46B 15/0034; A46B 15/0038; A46B 15/0053; A46D 1/0207; A61B 17/56; A61L 2/04; A61L 2/10; A61L 2/26; B08B 1/12; B08B 1/40; B08B 5/04; B08B 7/0057; B08B 7/0071; B08B 7/026; A46B 2200/1006; A61L 2202/11; A61L 2202/24

(57) ABSTRACT

An agitation device that uses a variety of mechanical, acoustic, electrical, and chemical media in order to effectively remove biofilm from an infected implant. The brush head embodiments include numerous shapes and sizes tailored for specific applications. Irrigation, suction, drug delivery and capture capabilities are also provided.

### RELATED APPLICATIONS

This application claims priority to U.S. Provisional Application Ser. No. 63/484,115 filed Feb. 9, 2023, entitled Anti-infection Surgical Device for Biofilm Agitation, incorporated herein by reference in its entirety.

### FIELD OF THE INVENTION

This invention relates generally to surgical devices, methods, and systems for use in surgical procedures to identify, disrupt, and evacuate microbial infections such as biofilm harbored on medical devices or implants and tissues in the human body.

### BACKGROUND

This invention relates generally to surgical devices, methods, and systems for use in surgical procedures to identify, disrupt, and evacuate microbial infections such as biofilm harbored on medical devices or implants and tissues in the human body.

Modern technology has powered the development of surgical implants, grafts and devices which improve the health and lifespan of patients suffering from various diseases and injuries. Over time, advancement in these medical devices has improved their ease of implantation into the human body as well as their biocompatibility. The lifecycle of such devices within the human body is increasing, and in some cases, the intent is for definitive or permanent implantation.

Despite the success of medical devices in terms of their ease of implantation, biocompatibility, lifespan, and efficacy as a therapeutic measure, they still present a serious risk for failure via infection. Medical implants are particularly susceptible to infections, because they are comprised of foreign materials such as non-native tissues, metals, or synthetic materials (e.g., human allografts, porcine, bovine grafts, stainless steel, cobalt-chromium, titanium, plastics, synthetic mesh, etc.) which can serve as a nidus for infections to take hold. Microbials such as bacteria and fungus often find these foreign implant surfaces desirable for attachment and growth. The microbials then proliferate and eventually disperse into the human body, potentiating and exacerbating infection (e.g., causing a state of septicemia or bacteremia). Medical device or implant infections can have devastating consequences for the patient as they greatly impact the morbidity and mortality of patients and skyrocket the healthcare costs associated with treating infection.

Orthopaedics is a consummate example of a medical field that frequently utilizes implants for patient care. Surgeons in this practice area use plates and screws for fractured bones, arthroplasty implants for knee and hip replacements, and spinal hardware such as rods and pedicle screws for vertebral support. While orthopaedic implants aim at improving health and function, they can become reservoirs for infections that compromise the patient's health. Bacteria involved in these infections can create biofilms-complex bacterial communities attached to the surfaces of implants. Biofilms have unique attributes including heightened adaptability and a protective Extracellular Polymeric Substance (EPS) that make them very difficult to treat with antibiotics alone.

The standard of care for orthopaedic implant infection consists of medical intervention with diagnosis (through various means including clinical history, examination, laboratory studies, etc.), resuscitation measures and antibiotic therapy. Medical intervention is often combined with a surgical intervention to combat infections. This type of intervention usually involves surgical exposure of the infection site to address the device-related infection via irrigation and debridement of necrotic and contaminated tissue. Due to established difficulty in removing biofilm from devices, surgical removal of the devices harboring the biofilm is often viewed as a necessary course of action to rid the host from infection. Unfortunately, these efforts may carry high morbidity for the patient resulting in increased blood loss, damage to surrounding soft tissue structures or neurovascular structures, bony compromise, or bone loss to the site—to list a few examples. These issues associated with device removal can have devastating consequences for a patient's overall health and function. Moreover, these medical and surgical management strategies often fail altogether, and the patient and healthcare workers must continue to wage a dire and costly effort—emotionally, physically, and financially—to eradicate or control device-related infections.

Device-related infections are not unique to the orthopaedic surgery specialty. All devices or implants carry the potential for infection, and that includes those used in the specialties of cardiology, general surgery, interventional radiology, ear nose throat (“ENT”), urology and plastic surgery, among others. In general surgery, interventions such as hernia repairs with prosthetic woven mesh can alleviate pain. In the cardiac space, implantable cardiac devices such as pacemakers and defibrillators have served as successful interventions for people with underlying cardiac and cardiovascular diseases to extend and enhance their life. There are numerous other examples of innovative medical devices, implants, grafts, and prosthesis that have greatly improved the quality and longevity of patients' lives. Despite the successes of medical engineering and application of these inventions in the surgical space, the possibility remains that a life-threatening infection will take hold of the implant as outlined above. Each surgical specialty must battle infections through various modes of prevention and treatment.

While the current standard of care requiring irrigation and debridement with possible removal of devices is occasionally effective in combating device-related infections, this method yields unpredictable and unsatisfactory results. In some instances, the risks associated with implant removal are so great that irrigation and debridement procedures are performed as the lone surgical strategy. The current surgical tools for device-related infection cases are limited and none effectively treat the devices themselves. Furthermore, no surgical instrument is outfitted with purposeful specifications or design elements that intentionally address biofilm agitation and removal.

However, there are a few products within the surgical setting which do attempt to eradicate the infection from the host but do so without specific biofilm-directed features. In the irrigation space, there are several solutions utilized by physicians to help wash away the infection (i.e., bacteria, fungus, etc. which may have formed biofilms). The most common solution employed is sterile saline. Sterile saline offers the advantage of an aqueous solution to rinse off debris and susceptible pathogens or to wash weak biofilm from implant surfaces with little risk of damage to the human tissues the solution(s) encounters. Unfortunately, infections with biofilm aggregating on the device surface rarely wash away from irrigation alone as the biofilms form secure attachments to the surfaces.

There are also several solutions and detergents which hold the capacity to kill or weaken pathogens they encounter. One such solution is a proprietary solution called Bactisure™ Wound Lavage which is an aqueous solution composed of ethanol, acetic acid, sodium acetate, benzalkonium chloride and water. Bactisure™ is used to remove debris as well as deconstruct biofilm's protective EPS. The destruction of the biofilm's EPS removes the bacteria or weakens them, making the bacteria more susceptible to medical intervention such as antibiotics or the patient's immune system. There are numerous iodine solutions or antiseptics which aim for a similar effect. While these detergents, soaps and antiseptics are effective measures in cleaning surgical infections, they also carry the risk of deleterious side effects to the patient, as they may be caustic to native human tissues. Furthermore, their handling, processing and removal often requires specific equipment and processes which can be cumbersome and expensive. As with sterile saline irrigation, these detergent or soap solutions often provide limited results as they do not penetrate nor disrupt the biofilm harbored on the medical device.

In terms of products which deliver irrigation solutions, there are several pulse irrigator or lavage devices on the market which are frequently used in infection cases due to their cleansing effect. Pulse irrigators, such as the Interpulse Irrigation System by Stryker, offers a powered irrigation device to deliver high to low pressures with the ability to exchange irrigation and suction spouts. The pulsatile irrigation may disrupt necrotic tissue and break up biofilm, removing them via suction from the surgical field. While irrigation solutions and the devices that deliver them have a role to play in the war against biofilms, well-documented studies have demonstrated that irrigation and suction alone are not enough to effectively remove biofilm from the surfaces of implants.

Still, measures such as scrub brushes or bristle canal brushes are mechanically deployed to physically remove or weaken biofilms and their protective EPS shield. These may mechanically disrupt biofilms or remove surrounding necrotic or contaminated tissues. However, they do not offer more sophisticated agitation of biofilms aided by technology. Additionally, these brushes are often not specific in their design to navigate and deliver mechanical scratching of the surgical site or device surface. Their use intraoperatively is inconsistent and not adopted in widespread practice protocols for infection surgical interventions. This is likely in part due to the inconsistent biofilm disruption that mechanical scratching alone provides, as well as the lack of product availability and education on the importance and challenges of biofilm disruption.

One device has recently emerged that is designed specifically for the purpose of mechanically removing biofilm. Biobrush™ is a powered brush that is being developed by OsteoRemedies® and consists of a rounded brush head that spins when attached to a powered shaft. However, the device does not appear to be small enough to clean joints percutaneously. Nor does the device include means of biofilm removal in addition to the mechanical rotation of the bristles over the surface of the implant.

There are also medical devices which feature technology such as sonication to disrupt biofilm. One such device is SonicOne which houses ultrasonic technology to debride unhealthy tissue for wounds or chronic infection sites. This innovative device readily lyses and removes bacteria and biofilms while attempting to preserve healthy tissue and vital structures such as nerves and vasculature. The device technology offers various attachments depending upon the type and topography of the wounds. While this is an innovative device, its focus is on addressing chronic wounds and it does not employ additional technologies like mechanical scratching features (e.g., a brush or bristles) nor advanced technology such as radiofrequency, electromagnetic wave therapy, or laser therapy. Additionally, this device is not designed for deep surgical wounds nor scaled to treat device surfaces.

Biofilms not only plague the medical industry, but they are also prevalent in the dental field. The dental industry has several technology-forward products aimed at cleansing the mouth and its associated structures (e.g., teeth, gums, etc.). Electric toothbrushes have an established use in disrupting dental plaque (i.e. dental biofilm) via sonication and mechanical scratching using brush heads. Philips Sonicare™ and Oral B Pro™ are two examples of at-home toothbrushes, which feature not only sonication and brush heads, but also incorporate additional user feedback technology such as a timer, pressure sensors, sonication mode control and companion apps. The brush heads are also replaceable.

Furthermore, scientific inquiry has led to the advent of radiofrequency (RF) strategies for disruption of biofilms. One such utilization in toothbrushes is the product, ToothWave™. This product combines sonication and radiofrequency technologies to clean biofilm from the mouth. There are also products in the dental space which mirror the intent of the surgical pulse irrigators described above. These mouth-cleansing devices have been developed for at-home use and operate specifically to penetrate the crevices between teeth, physically disrupting biofilm attached in hard-to-reach places. One such device is the WaterPik®. While these devices are effective for dental hygiene measures addressing plaque and other biofilms in the oral cavity, they lack many of the design specifications that would make them appropriate surgical instruments for the treatment of device-related infections. For instance, the brush head size, shape, and composition are not optimized for surgical fields or device surfaces. Finally, the aforementioned devices do not incorporate all of the technologies nor features outlined above (i.e. radiofrequency, sonication, bristles/nubs, interchangeable brush heads, handheld device with sterile packaging, suction, irrigation/solution applicator, etc.) into a single device or biofilm agitation system.

### SUMMARY

The present invention provides an improved configuration applicable to many clinical and surgical settings for addressing infections. This disclosure is generally directed to devices, methods and systems for irrigating and debriding (i.e., cleaning) surgical site infections. The concepts presented herein may be particularly suitable for treating and removing biofilm from the surfaces of implant-related infections and may combine mechanical scratching or brushing, sonication, radiofrequency, suction, and irrigation or dye application.

One aspect of the invention pertains to an electrosurgical device that effectively agitates biofilm adherent to medical devices, implants or tissues of the human body. The electrosurgical device is an instrument equipped with sonication (i.e., various sonication frequencies or energy including ultrasonication) and radiofrequency technologies. The electrosurgical device is a wand or handheld construct with contoured surfaces and edges allowing for ease of use and control. This is an important consideration as device handling under surgical conditions may be challenging given the aqueous or slippery environment(s) in addition to the user frequently wearing surgical gloves. The device's sonication and radiofrequency delivery technologies are positioned at the front end of the device and delivered to the surface being treated through an attachment, such as a disposable attachment in the form of a brush head.

Another aspect of the invention provides numerous designs for brush head attachments. Generally, the brush head includes bristle elements, radiofrequency emitter(s) and/or a sonicator electronic element. The brush head is an attachment piece that may be readily exchanged for different brush heads featuring various sizes, shapes, and brush head designs specifically purposed to access and navigate the intended surgical field and device surface(s). This removable brush head will allow the user to select the most advantageous brush head design to navigate specific exposure situations, implant specifications and human tissue characteristics. The backside of the brush head may also feature additional bristle heads, sweeper or squidge, agitation or scraping nubs to further benefit the mechanical agitation and removal of unwanted microbial biofilm, debris, etc.

Another aspect of the invention is a tubing conduit for the delivery of irrigation and application of solution which may pulse or be delivered under various high or low pressures to wash away and disrupt biofilm. Additionally, this channel can deliver specialized solutions or dyes which may identify and attach to biofilm, thus creating (1) a target at which the user can direct the disclosed invention and (2) feedback for adequate biofilm removal. The device possesses a vacuum suction channel to be utilized for evacuating irrigation, solution, unwanted debris, serous and sanguineous drainage, purulence, etc. The irrigation channel and suction may be positioned at the underbelly of the device at its more distal portion.

In one aspect of the invention, the body of the device features buttons and dials which control the powering on/off as well as the setting levels (e.g., high, medium, low; various numerical values) for the sonication of the bristle heads, radiofrequency, and the irrigation channel and suction elements. The device, in at least one embodiment, is powered electronically and may be plugged into a specific power box in addition to an irrigation bag pump and suction tank(s).

Another embodiment of the invention provides a device that is manufactured for single-use versions powered by battery. The device will be processed in a sterile fashion and housed in easy-to-open packaging to allow for sterile opening of the device for use in the sterile surgical field.

Still another embodiment provides single-use power station that may be battery powered or may plug into an outlet to access mains power.

Additional embodiments may include, but are not limited to:
laser technology to synergize with sonication and radiofrequency measures to disrupt biofilm;
a lighting system to identify and illuminate biofilm presence on implant surface (may be utilized in conjunction with a specific applicator/solution or dyes which adhere to the biofilm—lighting may include ultraviolet frequencies);
a timer feedback to alert the user on various specific time periods of use—this feedback may be featured in a lighting system or felt mechanically by the user via pulse/vibration (e.g., light may illuminate or device pulses/stops sonication when two minutes have been reached, or 30 second time points have been reached);
a pressure sensor which may alert the user when there is too great or too little pressure being applied to the device which may negatively affect the optimized pressure for bristle/brush head delivery of sonication, radiofrequency or mechanical scratching;
a bar barrier on the front side of the brush head which helps the user gauge the distance from the implant or directed surface the invention is treating to ensure the optimal contact or distance is met (or not surpassed) to deliver sonication, radiofrequency or mechanical scratching;
a thermal application at the brush head to either heat or cool the implants;
various layouts of brush head and associated bristles/nubs/squidge;
a reusable portion of the invention (e.g., reusable power source), or an entirely reusable version of the invention;
various configurations, shapes, and sizes of the brush heads, handheld wand, and power source;
various configurations, shapes, design, color and sizes of the entire device;
additional power sources (e.g., AC power extension, chemical reactions, and non-battery-operated power sources);
additional mechanisms in addition to buttons or dials for initiating power source (e.g., button turn on and off, switch, etc.);
various configurations, shapes, sizes and locations of sonication, radiofrequency emitters or beacons;
a packaging configuration that allows for sterile packaging to ensure device is sterile upon opening; and
a packaging configuration that allows for the potential to turn device on while still contained in the packaging (i.e., allows for device to be turned on and operated in the sterile environment through packaging).

### BRIEF DESCRIPTION OF THE DRAWINGS

Referring to the drawings, wherein like numerals represent like parts throughout the several views:

FIG. 1 is a side elevation of an embodiment of an agitation device of the invention;

FIG. 2 is a side elevation of an embodiment of an agitation device of the invention;

FIG. 3 is a plan view of an embodiment of an agitation device of the invention;

FIG. 4 is a side elevation of an embodiment of an agitation device of the invention;

FIG. 5 is a side elevation of an embodiment of a brush head for use with an agitation device of the invention;

FIG. 6 is a front view of an embodiment of a brush head for use with an agitation device of the invention;

FIG. 7 is a front view of the brush head of FIG. 6 showing the motion thereof after having been activated;

FIG. 8 is a front view of an embodiment of a brush head for use with an agitation device of the invention;

FIG. 9 is a side elevation of an embodiment of a brush head for use with an agitation device of the invention;

FIG. 10 is a side elevation of an embodiment of a brush head for use with an agitation device of the invention;

FIG. 11 is a side elevation of an embodiment of a brush head for use with an agitation device of the invention;

FIG. 12 is a side elevation of an embodiment of a brush head for use with an agitation device of the invention;

FIG. 13 is a side elevation of an embodiment of a brush head for use with an agitation device of the invention;

FIG. 14 is a side elevation of an embodiment of a brush head for use with an agitation device of the invention;

FIG. 15 is a side elevation of an embodiment of a brush head for use with an agitation device of the invention;

FIG. 16 is a side elevation of an embodiment of a brush head for use with an agitation device of the invention;

FIG. 17 is a side elevation of an embodiment of a brush head for use with an agitation device of the invention;

FIG. 18 is a side elevation of an embodiment of a brush head for use with an agitation device of the invention;

FIG. 19 is a front view of an embodiment of a brush head for use with an agitation device of the invention;

FIG. 20 is a side elevation of an embodiment of a brush head for use with an agitation device of the invention;

FIG. 21 is a side elevation of an embodiment of a brush head for use with an agitation device of the invention;

FIG. 22 is a side elevation of an embodiment of a brush head for use with an agitation device of the invention;

FIG. 23 is a side elevation of an embodiment of a brush head for use with an agitation device of the invention;

FIG. 24 is a side elevation of an embodiment of a brush head for use with an agitation device of the invention;

FIG. 25 is a side elevation of an embodiment of a brush head for use with an agitation device of the invention;

FIG. 26 is a side elevation of an embodiment of a brush head for use with an agitation device of the invention;

FIG. 27 is a front view of an embodiment of a brush head for use with an agitation device of the invention;

FIG. 28 is an end view of an embodiment of a brush head for use with an agitation device of the invention;

FIG. 29 is a side elevation of an embodiment of a brush head for use with an agitation device of the invention;

FIG. 30 is a side elevation of an embodiment of a brush head for use with an agitation device of the invention;

FIG. 31 is a side elevation of an embodiment of a brush head for use with an agitation device of the invention;

FIG. 32 is a side elevation of an embodiment of a brush head for use with an agitation device of the invention;

FIG. 33 is a side elevation of an embodiment of a brush head for use with an agitation device of the invention;

FIG. 34 is a diagram of an embodiment of a control system according to the invention;

FIG. 35 is a diagram of an infected knee implant with a magnified area showing an infection;

FIG. 36 is a diagram of the infected knee implant of FIG. 35 being treated according to the invention;

FIG. 37 is a diagram of the knee implant of FIG. 35 after treatment according to the invention with a magnified area showing the efficacy of the treatment;

FIG. 38 is a diagram of a knee implant being treated percutaneously using a device of the invention;

FIG. 39 is a diagram of a spinal implant being treated with an embodiment of an agitation device of the invention;

FIG. 40 is a diagram of an ankle plate being treated with an embodiment of an agitation device of the invention; and,

FIG. 41 is a diagram of an electronic cardiac implant being treated with an embodiment of an agitation device of the invention.

### DESCRIPTION

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

### Handle Embodiments

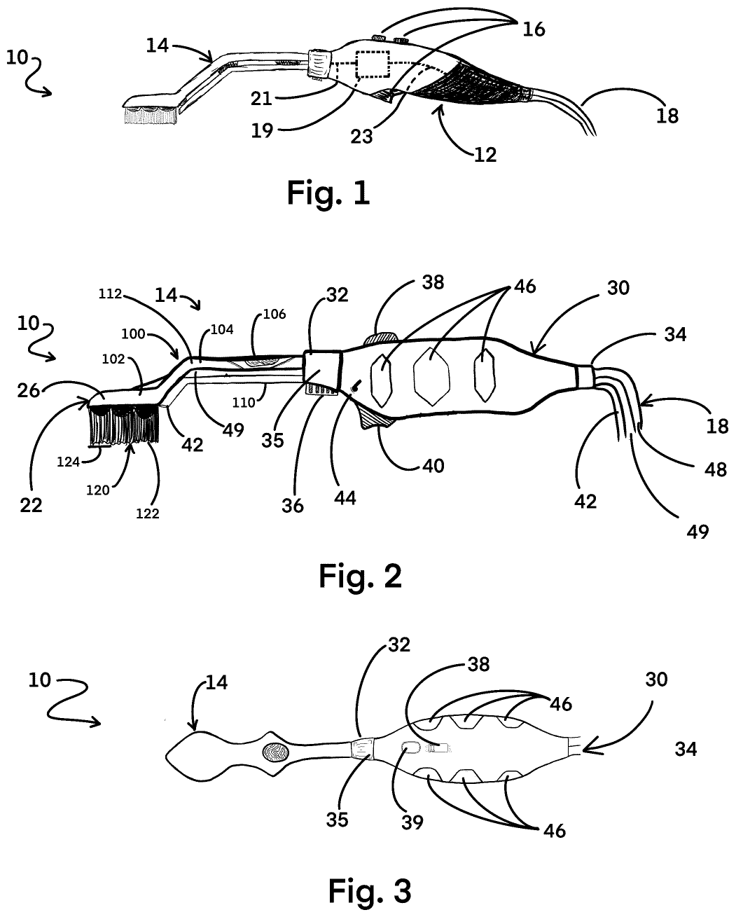

Referring now to the Figures, and first to FIG. 1, there is shown an embodiment of an agitation device 10. Generally, each agitation device discussed herein has a handle 12 and a tool 14, typically in the form of a brush head. The various embodiments of handles and tools will be numbered independently and, for purposes of clarity, general reference to handles will be denoted as handle 12 and general reference to a brush heads or other attachments will be denoted as tool 14.

Each handle 12 may include at least one control, numbered in a general sense as control 16, and a supply cord 18, that may provide power, fluid, suction, and/or other forms of energy such as UV light, for example. In at least one embodiment, the handle 12 is designed as a non-disposable component to reduce waste. In this regard, the handle 12 is constructed to be easy to sanitize and is not to be inserted into the patient. Disposable plastic sleeves, not shown, may further be provided to prevent contaminants from contact the treatment site. Alternatively, the handle 12 may be intended as a single-use device and may be powered by a battery. Single-use embodiments may be supplied with or without the tool 14 and will be processed in a sterile fashion and presented in a sterile, easy to open package that allows for the sterile opening of the device for use in the sterile surgical field.

Inside the handle 12 is housed an agitating mechanism 19 used to animate the agitating head 22. The agitating mechanism 19 converts an electrical current and/or signal from an electrical lead 23 to a mechanical motion, which is then translated to the tool 14 via a shaft 21. Examples of agitating mechanisms include, but are not limited to; motors, piezoelectric transducers, electromagnetic oscillators, etc.

FIGS. 2 and 3 provide a side elevation and a plan view, respectively, of a first embodiment of an agitation device 10. The agitation device 10 includes a handle 30 having a distal end 32 and a proximal end 34. At the distal end 32 is a locking connector 35 usable to attach and a lock a tool 14 to the distal end 32 of the handle 30. The locking connector 35 is shown as including a release button 36 that, when depressed, allows a tool 14 to be released from the handle 30. The locking connector 35 may be provided as a component of the handle 30, such that when the tool 14 is detached, the locking connector 35 remains with the handle 30. Alternatively, the locking connector 35 may be provided as a component of the tool 14, as would be realized by one skilled in the art.

The handle 30, proximal of the distal end 32, includes an on/off control 38 positioned to be operable by the thumb of a user when gripped, and a corresponding power indicator light 39 just distal of the off control 38 such that the corresponding power indicator light 39 is visible even when a thumb is covering the off control 38. A trigger control 40 is provided and positioned for activation by the user's forefinger. The trigger control 40 is constructed to open and close an irrigation supply lumen 42 provided by the supply cord 18. A trigger lock 44 is also provided to allow the trigger 40 to be released without closing the irrigation supply lumen. One or more grip surfaces 46 may be provided to improve handling of the agitation device 10.

The proximal end 34 of the handle 30 is connected to a supply cord 18. The supply cord 18 includes the aforementioned irrigation supply lumen 42, which leads to a source of irrigation fluid (not shown). The irrigation fluid may be any fluid known to one skilled in the art and may include antibiotics or other medicaments or agents usable to aid in the removal and/or prevention of the reemergence of biofilms.

The supply cord 18 further includes an electrical cable 48 and a suction lumen 49 for removal of the irrigation fluid and blood and other matter as necessary to prevent fluid buildup and/or obstruction of visualization componentry, if applicable.

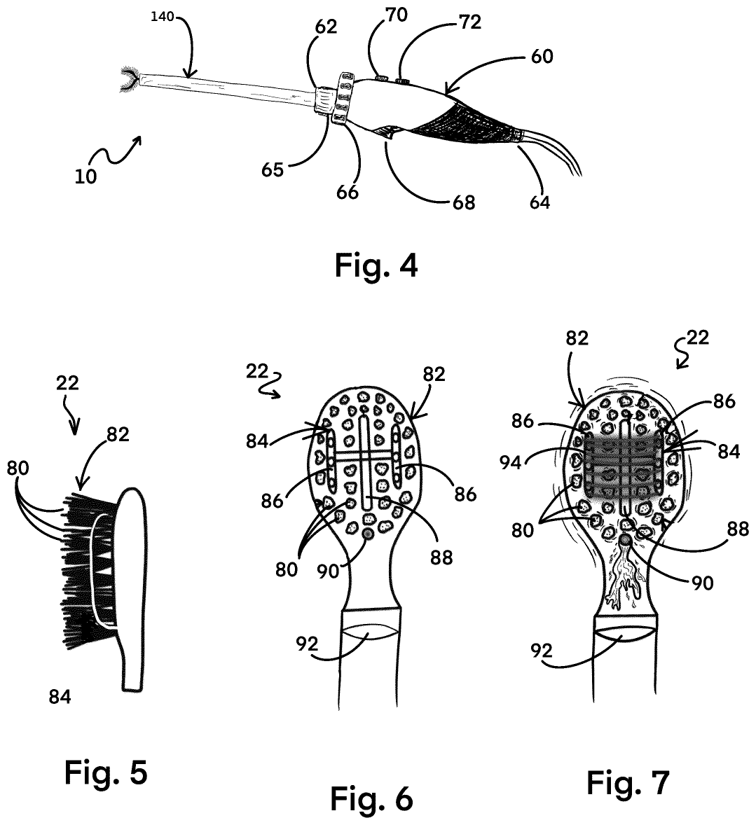

FIG. 4 provides another embodiment of an agitation device 10 that includes a handle 60 having a distal end 62 and a proximal end 64. At the distal end 62 is a locking connector 65 usable to attach and a lock a tool 140 to the distal end 62 of the handle 60. The locking connector 64 is shown as including a threaded collar 66 that prevents unintentional disengagement of the tool 140 from the handle 60. The locking connector 65 may be provided as a component of the handle 60, such that when the tool 140 is detached, the locking connector 65 remains with the handle 60. Alternatively, the locking connector 65 may be provided as a component of the tool 140, as would be realized by one skilled in the art. The handle 60 further includes an irrigation trigger 68 and thumb controls 70 and 72 provided for such functions as power control and auxiliary functions, for example, suction control.

### Tool Features

Each tool 14 will include a body 20 for attaching the tool 14 to the handle 12. The tool 14 further includes an agitating head 22 that uses various components such as brush bristles, one or more picks, or other contact surfaces as will be described in detail below. In at least one embodiment, the tool 14 is a disposable component.

It is to be further understood that, for purposes of brevity, the various tool 14 embodiments and the various handle 12 embodiments, may be used interchangeably to the extent that the handle 12 is configured for compatibility with the modalities being utilized by the tool 14, as would be understood by one skilled in the art. It is further to be understood, that combinations of the various agitating head 22 embodiments described herein may be combined or separated on a given tool 14 without departing from the spirit of the invention.

FIG. 2 further shows an embodiment 100 of a tool 14 having a body 102 and an agitating head 22 in the form of a brush 120. The body 102 has a top 104 that includes a textured finger pad 106 to aid in control of the device. An underside 110 of the tool embodiment 100 includes connectable extensions of the irrigation supply lumen 42 and suction lumen 49 that follow the 102 and terminate at the brush 120. The body 102 may be shaped to form an angle 112 to assist in providing a desirable access to the surface to be cleaned. As will be seen, a variety of body shapes are provided for various applications. These shapes include angles of varying degrees and curves of varying shapes and radii. The bodies terminate in a rigid terminus 26 that forms a part of the agitating head 22, namely that part of the head that houses various cleaning features and to which the bristles, mounds, sponges, and other components are attached. The rigid terminus 26 may have a variety of shapes and angles relative to the rest of the body 20 as will be seen below.

The embodiment 100 includes a brush head 120 to agitate the biofilm. The brush head 120 includes a plurality of bristles 122 arranged in a parallel configuration and sized to create a flat brushing surface 124. When the handle 30 is activated, a vibration device (not shown) housed within the handle 30 causes the brush 120 to vibrate rapidly such that the user does not have to move the 120 back and forth over a surface to remove the biofilm, which could cause user fatigue and unintended injury to the patient. The plurality of bristles 122 in this embodiment 100, as well as the many other embodiments of agitating head 22 designs described herein, may be formed of materials such as wire, synthetics (e.g. nylon), rubber, natural fibers and other materials as are known in the art and suitable for use in a medical setting.

FIGS. 5-8 show an example of an agitation head 22 having various agitation features that may be provided on any of the heads described herein alone or in any combination. As discussed previously, the agitating head 22 may include a plurality of bristles 80 that combine to form a brush 82 having varying shapes as will be described below. Additionally, the agitating head 22 is shown has including a radio frequency (RF) emitter 84. The emitter 84 is shown as including two RF elements 86 separated by a barrier bar 88. The barrier bar 88 helps the user gauge the distance from the implant or directed surface the invention is treating to ensure the optimal contact or distance is met (or not surpassed) to deliver sonication, radiofrequency or mechanical scratching.

As best seen in FIGS. 6 and 7, an irrigation port 90 is located just proximal of the plurality of bristles 80 to provide such liquids as irrigation fluid to keep the procedural site clear of dislodged biofilm particles during the cleaning process, cleaning agents, anti-bacterial or other medicaments, and/or dyes to aid in visualization of the cleaning coverage. Proximal of the irrigation port 90 is a suction port 92 used to remove the debris and to prevent fluid buildup at the site.

FIGS. 6 and 7 are distinguished by the depiction of motion in the bristles 80 in FIG. 7, represented by the blurred area 94. As discussed throughout, bristle motion is a component of the invention that aids in biofilm disruption and can be provided to the bristles using vibration, rotation, oscillation, sonication, etc.

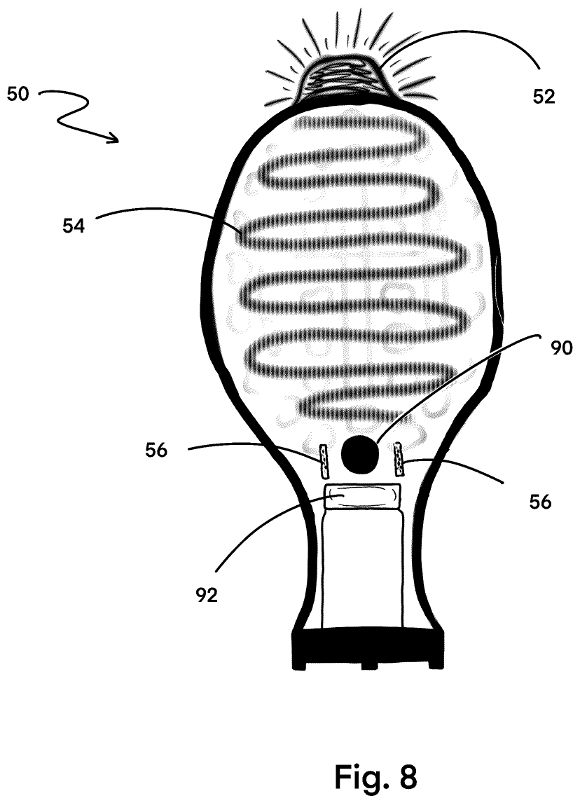

FIG. 8 shows an agitation head 50 having additional features. The agitation head 50 has at a distal end thereof an ultraviolet (UV) light source 52. Embedded within the head is a thermal element 54 that can be used to heat or cool the head using thermal technologies such as infrared (IR), resistive coils, cryotherapy lumens, and the like. Electromagnetic elements 56 are also shown and may be used to deliver electromagnetic waves to the implant being cleaned. Also shown is an irrigation port 90 and a suction port 92.

### Agitating Heads

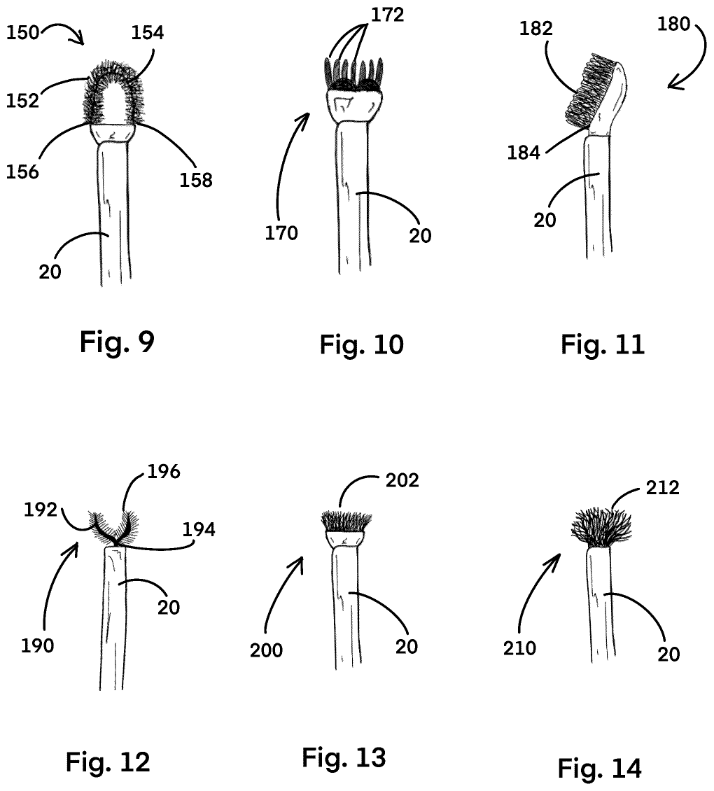

FIGS. 9-30 show a variety of non-limiting examples of agitating heads 22 that may be used with one or more of the handle 12 embodiments of the invention. It is to be understood that FIGS. 9-30 are provided to depict various agitating head 22 shapes, and each of these heads may include sonication, irrigation, suction, vibration, RF energy, light energy, and the like in order to enhance the biofilm removal efficacy of the tool 14. It is further to be understood that each of the agitating head 22 depicted herein may be provided in a variety of sizes such that the agitating head 22 may be ideally paired with a given procedure and patient.

FIG. 9 shows an agitating head 150 that utilizes a cylindrical brush formed from bristles 152 radiating from a wire 154. The wire 154 has a first end 156 and a second end 158. The wire 154 is curved to form a U-shape, allowing each of the first end 156 and the second end 158 to be attached to the distal end of the tool body 20.

FIG. 10 shows an agitating head 170 that includes a plurality of elastomeric fingers 172 extending distally from the distal end of the body 20.

FIG. 11 shows an agitating head 180 that is angled and includes a plurality of relatively parallel bristles 182 extending normally relative to an angled surface 184 of the agitating head 180 to which the plurality of relatively parallel bristles 182 are attached.

FIG. 12 depicts an agitating head 190 that has is formed of a twisted pair of wires 192 joined at a proximal end 194 for connection to the body 20 and separating in a distal direction to form a U shape. Each of the wires 192 has a plurality of bristles 196 radiating outwardly from the wires 192 to form a brush.

FIG. 13 shows an agitating head 200 that includes a plurality of relatively parallel bristles 202 extending distally from the distal end of the body 20.

FIG. 14 shows an agitating head 210 that has a plurality of radiating bristles 212 extending from the distal end of the body 20 to form a tuft.

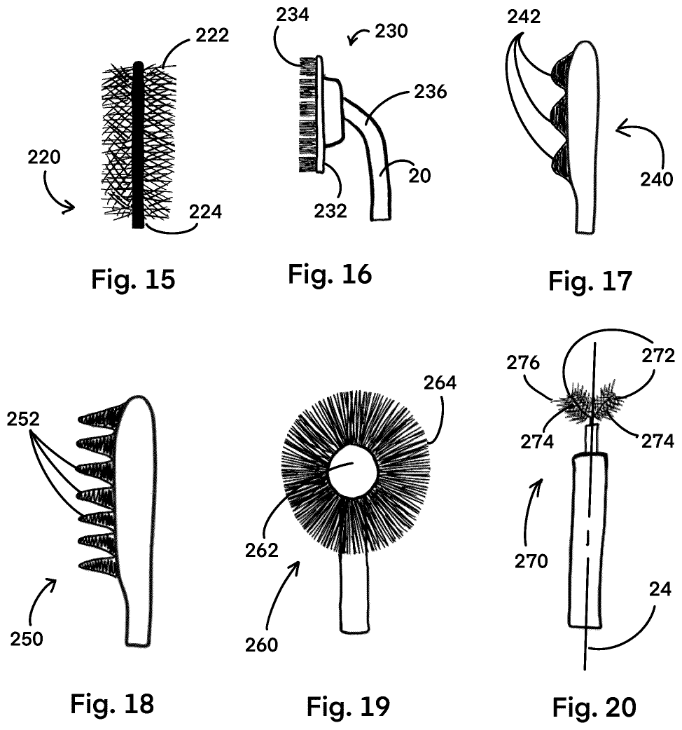

FIG. 15 shows an agitating head 220 that includes a tube brush 222 with bristles radiating outwardly from a twisted wire core 224. In at least one embodiment, the handle 12 to which the agitating head 220 is attached, has a rotating, oscillating, and/or vibrating motion to the agitating brush 222.

FIG. 16 shows another embodiment of an agitating head 230, which has a disc-shaped plate 232 attached to a body 20, which may have a curve 236 of varying radii, and a plurality of relatively parallel bristles 234 extending from either the perimeter of the plate or the entirety of the plate 232. The plate 232 rotates, oscillates and/or vibrates relative to the body 20 when energized by a handle 12.

FIGS. 17 and 18 provide agitating heads with elastomeric or rubber features like that of the embodiment shown FIG. 10. The agitating head 240 of FIG. 17 has a plurality of mounds 242 that may allow the user to place increased pressure on the surface being cleaned. This embodiment may additionally provide tactile feedback to the user as rubber-biofilm interface tends to be slippery while rubber tends to rub or “squeak” against a wet, clean surface. Thus, the user may be able to feel or even hear the transition between the two sensations during the cleaning process. The embodiment of FIG. 18 provides an agitating head 250 with a plurality of rubber or elastomeric conical extensions 252, which are longer and more flexible than the mounds 242, thus being closer in similarity to the plurality of elastomeric fingers 172 of the embodiment shown in FIG. 10.

FIG. 19 shows another embodiment of an agitating head 260, which has a disc-shaped plate 262 attached to a body 20, and a plurality of relatively radial bristles 264 extending from the perimeter of the plate 262 to form a brush roller. The plate 262 rotates, oscillates and/or vibrates relative to the body 20 when energized by a handle 12.

FIG. 20 depicts an embodiment of an agitating head 270, which has a plurality of small, angled brushes 272 extending from a distal end of the body 20. Each of the plurality of brushes 272 has a twisted wire core 274 that is angled relative to a longitudinal axis 24 of the body 20 between 0 degrees and 90 degrees to the longitudinal axis 24, but more preferably between 20 degrees and 70 degrees to the longitudinal axis 24. Each twisted wire core 274 has a bristles 276 radiating outwardly from the twisted wire core 274. The angled brushes 272 rotate, oscillate and/or vibrate as a single unit relative to the body 20 when energized by a handle 12.

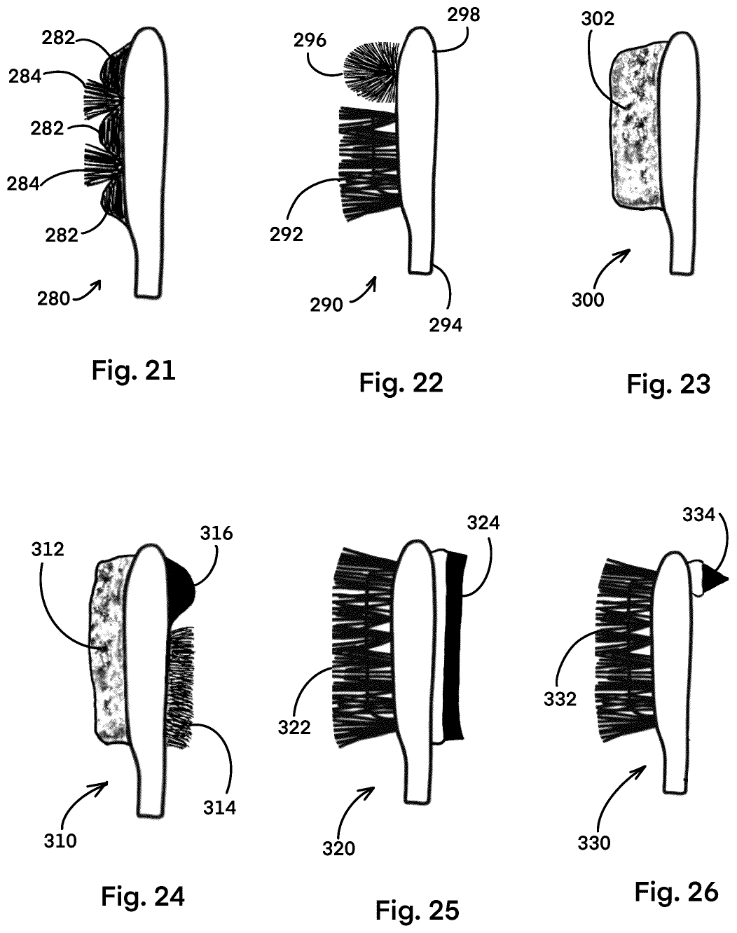

FIG. 21 shows an embodiment of an agitating head 280 that uses a combination of agitators. Provided are three spaced-apart rubber or elastomeric mounds 282, similar to the agitating head 240 of FIG. 17. Between the adjacent elastomeric mounds 282 are concentrations of bristles 284 extending beyond the elastomeric mounds 282 such that the bristles 284 contact the surface being cleaned before the elastomeric mounds 282 make contact.

FIG. 22 is an embodiment of an agitation head 290 that has a uniform set of bristles 292 near a proximal end 294 of the agitation head 290, similar to the bristle configuration of a toothbrush, and a hemispherical tuft of bristles 296 near a distal end 298 of the agitation head 290.

FIG. 23 depicts an embodiment of an agitation head 300 that uses a sponge 302 as an agitating medium. The sponge may include an anti-bacterial or other medicament, a bio-compatible surfactant or cleaning agent, and may be absorbent such that biofilm is captured by the sponge 302.

FIG. 24 provides another example of how two or more of the agitating media described herein can be combined. There is depicted an agitation head 310 that includes a sponge 312 on one side of the agitation head 310, and a brush 314 and rubber mound 316 on the other side of the agitation head 310.

FIG. 25 shows an embodiment of an agitating head 320 that has a brush 322 on one side and a wiper 324 on the other side. The wiper 324 may be constructed of a biocompatible elastomer or rubber material.

FIG. 26 shows an embodiment of an agitating head 330 that has a brush 332 on one side and a rubber or elastomeric cone 334 on the other side.

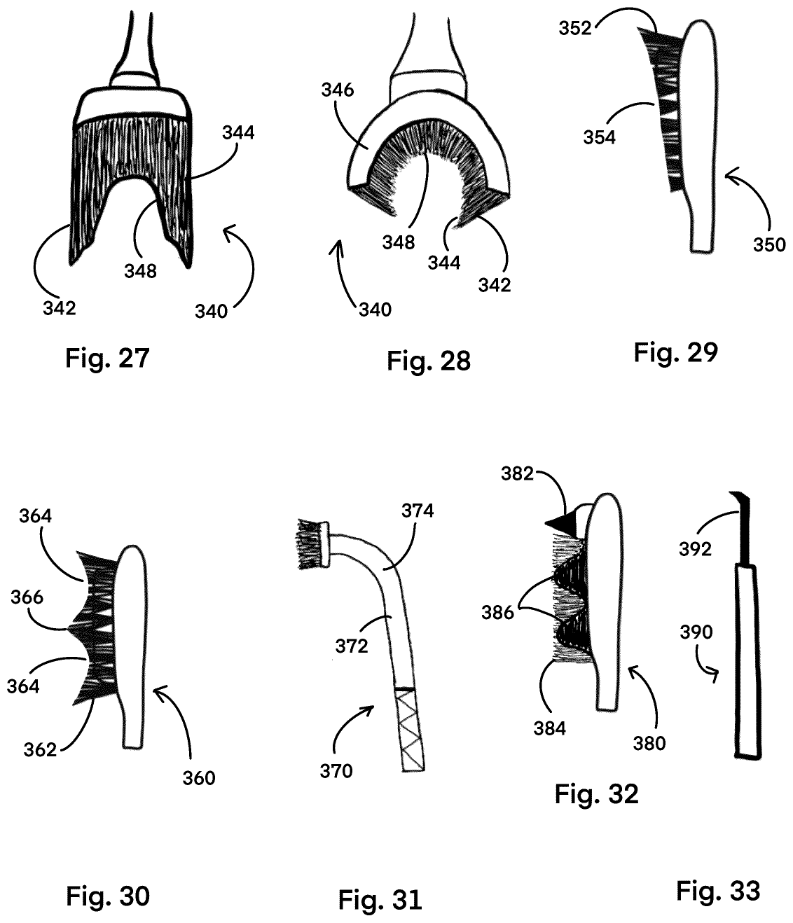

FIGS. 27 and 28 depict a non-limiting example of an embodiment of an agitating head 340 with a brush 342 that is shaped for a specific task. The brush 342 has bristles 344 that extend in a parallel fashion from a U-shaped element 346. To add further use-customization to the brush 342, the bristles 344 are of varying lengths and arranged such that the distal working ends of the bristles 344 combine to form an arc 348. This brush 342 may be ideally suited to clean rod-shaped implants or implants with crevices.

FIGS. 29 and 30 depict additional embodiments of agitating heads that use brushes with bristles that are sized and arranged to form contoured working surfaces. The embodiment of an agitation head 350 shown in FIG. 28 has bristles 352 sized and arranged to form a sloping working surface 354. FIG. 29 shows an agitating head 360 with bristles 362 sized and arranged to form a working surface comprising a pair of juxtaposed arcuate sections 364 that form a vertex 366 therebetween useful for extending between adjoining surfaces of an implant.

FIG. 31 depicts an embodiment of an agitating head 370 that has a flexible body 372 with a controllable curve 374. The controllable curve 374 is adjustable using a control on the handle that activates a steering mechanism, such as a steerable guidewire, as is known in the art.

FIG. 32 depicts an embodiment of an agitating head 380 with a distal rubber cone 382 and bristles 384 proximal of the rubber cone 382. Two mounds 386 are nestled within the bristles 384.

FIG. 33 depicts an embodiment of an agitating head 390 that provides a pick 392 instead of a brush.

### Control System

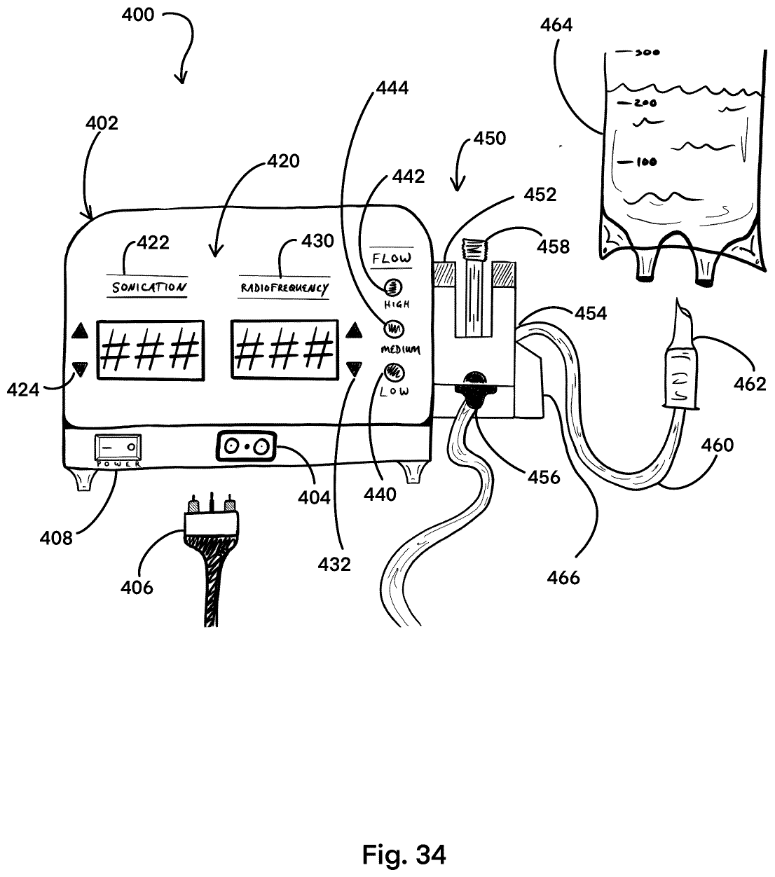

FIG. 34 shows an example of a control system 400 for use in powering and controlling the various embodiments of the agitation device 10 of the invention. The control system 400 generally includes a controller 402 with a display 420, and an irrigation/suction system 450. The controller 402 includes a power outlet 404 for accepting a plug 406 from a power supply. Adjacent the power outlet 404 is an on/off switch 408 for controlling the power being supplied to the agitation device 10.

The display 420 provides information and controls related to sonication and radiofrequency. A sonication readout 422 displays a selected sonication level. Sonicator selector buttons 424 are located next to the sonication readout 422 that allow the user to set the sonicator to a desired frequency. Similarly, an RF readout 430 displays a selected RF frequency and has located next to it, RF selector buttons 432 that allow the user to select a desired frequency.

Also included on the controller 402 are irrigation level selectors 440, 442 and 444 that allow the user to select varying flow rates for the irrigation/suction system 450, which is attached to the side of the controller 402. The irrigation/suction system 450 includes a pump 452, such as a diaphragm pump, with an inlet port 454 and an outlet port 456. The pump 452 may include a pump handle 458, usable to prime the pump, if necessary, prior to energizing the system.

The inlet port 454 is connected to a supply line 460 that has a distal end featuring a spiked connector 462 used to establish fluid flow communication with a source of irrigation fluid, in this case an irrigation solution bag 464. The outlet port 456 connects to the irrigation supply lumen 42.

The irrigation/suction system 450 may also be configured with a fluid sample collection tap 466 connected to the suction lumen 49. The fluid sample collection tap 466 allows a sample to be taken during a procedure. The fluid sample collection tap 466 may be opened to allow a slow, controlled trickle of fluid to fill a vial so that it may be sent to a laboratory for bacterial identification and other testing.

### Methodology

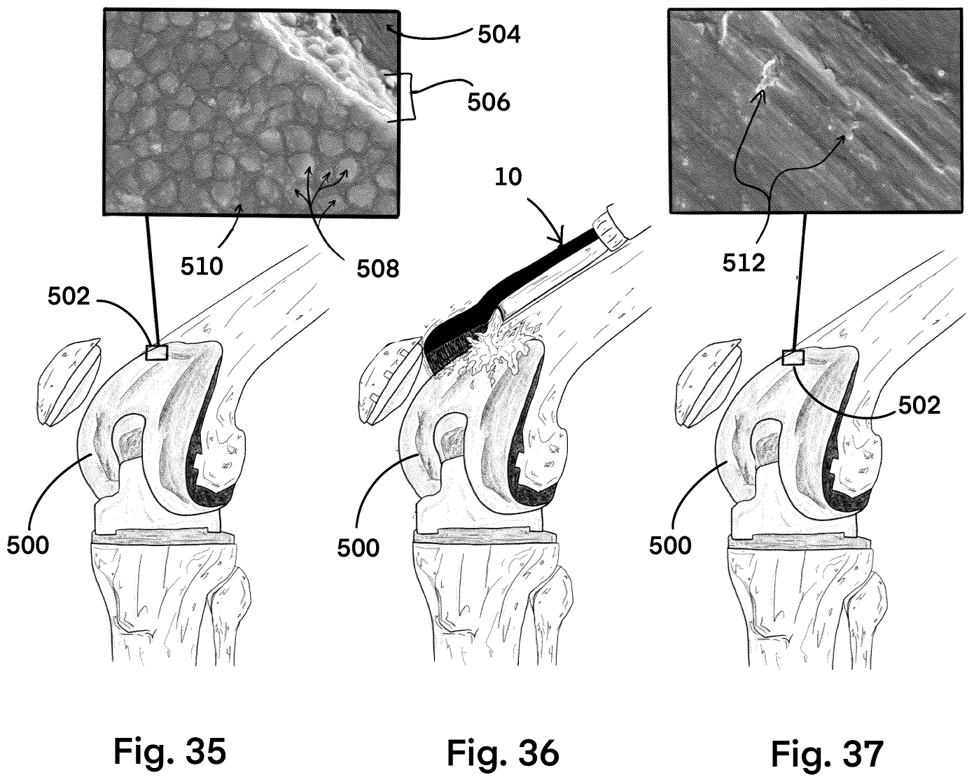

FIGS. 35-41 show examples of how the agitation device 10 of the invention has been used on infected implants with positive results. FIG. 35 shows a knee implant 500 that is infected with a biofilm. An area 502 of the knee implant 500 is expanded to show microscopic detail. In the upper right corner of the magnification, there is shown an implant/human tissue surface 504 that has not yet been infected, as a reference. Below the human tissue surface 504, the depth of the biofilm layer is apparent at 506. The surface of the biofilm shows that it is made up of individual bacteria 508, such as Staphylococcus aureus, in a biofilm state. Between the individual bacteria 508 exists an extracellular polymeric substance (EPS) or an extracellular matrix (ECM) 510.

FIG. 36 shows the knee implant 500 being treated with an agitation device 10. In this example, the agitation device 10 was used to apply mechanical scratching and brushing using sonication and RF to the knee implant 500. Irrigation with a dye was also used.

FIG. 37 shows the results of the treatment. The same area 502 was again magnified and showed that the biofilm had been removed at 512.

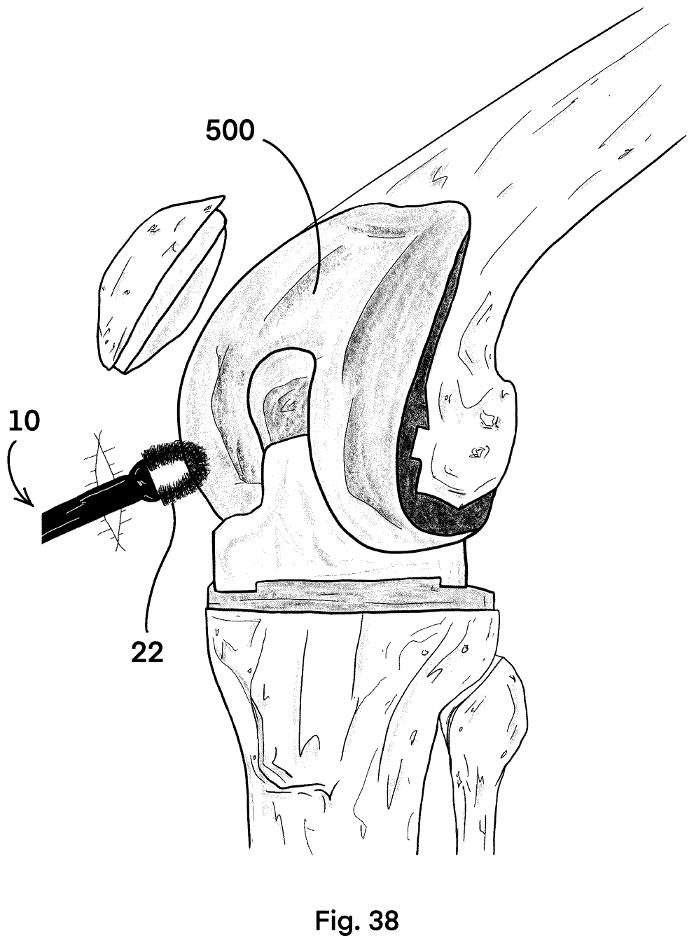

FIG. 38 shows the knee implant 500 being treated percutaneously using an agitation device 10 with a small agitating head 22.

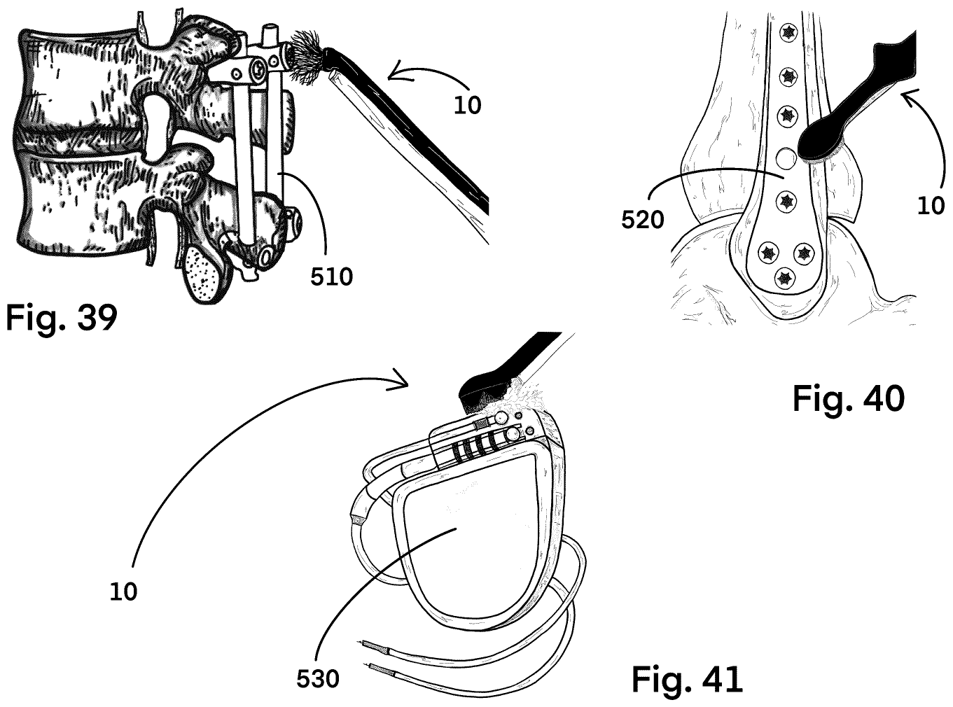

FIG. 39 shows an embodiment of an agitation device 10 being used to clean a spinal implant 510.

FIG. 40 shows an embodiment of an agitation device 10 being used to clean an ankle plate 520.

FIG. 41 shows an embodiment of an agitation device 10 being used to clean an electronic cardiac implant 530.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof. Similarly, the scope of the following claims should be interpreted according to the knowledge of one skilled in the art and the conventions and practices used thereby in light of the drawings and descriptions above.

What is claimed is:

1. An agitation device for cleaning implants comprising:
a handle having a proximal end and a distal end, the handle including;
at least one control;
an agitating mechanism within the handle;
a supply cord attached to the proximal end of the handle;
a tool attached to the distal end of the handle, the tool including:
a body;
an agitating head at a distal end of the body that becomes animated when the agitating mechanism is energized;
an irrigation supply lumen extending from a source of irrigation fluid through the supply cord, through the handle, and along the body such that irrigation fluid may be supplied to a site of the implant being cleaned by the agitating head.

2. The agitation device of claim 1 further comprising a suction lumen extending from a through the supply cord, through the handle, and along the body such that irrigation fluid may be removed from the site of the implant being cleaned by the agitating head.

3. The agitation device of claim 1 wherein the agitating head comprises an ultraviolet light source.

4. The agitation device of claim 1 wherein the agitating head comprises a plurality of bristles.

5. The agitation device of claim 1 wherein the agitating head comprise an elastomeric conical extension.

6. The agitation device of claim 1 wherein the agitating head comprises a radiofrequency transmitter.

7. The agitation device of claim 1 wherein the agitating head comprises a plurality of bristles and rubber mounds.

8. An agitation device for cleaning implants that uses a plurality of mechanisms for removing biofilm comprising:
a supply cord constructed to supply electricity and at least one of fluid and suction;
a handle connected at a proximal end to the supply cord;
a tool connected to a distal end of the handle;
a first mechanism that causes the tool to remove biofilm from an implant when used thereon, the first mechanism including an agitating mechanism within the handle that animates the tool when energized by the electricity from the supply cord such that the tool mechanically agitates biofilm on the implant when the tool contacts the implant;
a second mechanism that assists the tool in removing biofilm from an implant when used thereon.

9. The agitation device of claim 8 wherein the second mechanism comprises irrigation fluid applied to the biofilm with the tool.

10. The agitation device of claim 9 further comprising a suction lumen that removes the irrigation fluid and agitated biofilm from the implant.

11. The agitation device of claim 10 further comprising a fluid sample collection tap associated with the suction lumen usable to capture fluid samples.

12. The agitation device of claim 8 wherein the second mechanism comprises a thermal element.

13. The agitation device of claim 8 wherein the second mechanism comprises electromagnetic elements.

14. The agitation device of claim 8 wherein the second mechanism comprises ultraviolet light.

15. The agitation device of claim 8 wherein the second mechanism comprises radiofrequency emitters.

16. A method of removing biofilm from an implant comprising:
contacting the implant with an agitation device having at least one agitation mechanisms;
energizing the agitation device to cause the at least one agitation mechanism to oscillate, vibrate, rotate or sonicate a tool at a distal end of the agitation device;
moving the tool across the implant until the biofilm is removed;
utilizing at least a second mechanism of the agitation device, in addition to the agitation mechanism, to remove the biofilm.

17. The method of claim 16 wherein utilizing the at least a second mechanism of the agitation device comprises applying ultraviolet light to the biofilm.

18. The method of claim 16 wherein utilizing the at least a second mechanism of the agitation device comprises irrigating the biofilm.

19. The method of claim 16 wherein utilizing the at least a second mechanism of the agitation device comprises applying suction to the biofilm.

20. The method of claim 16 wherein utilizing the at least a second mechanism of the agitation device comprises radiating the biofilm.
